(19) United States
(12) Patent Application Publication
(10) Pub. No.: US 2008/0234646 A1
(43) Pub. Date: Sep. 25, 2008

(54) Odour-Preventing, Water-Absorbing Compositions

(76) Inventors: Volker Braig, Weinheim-Lutzelsachsen (DE); Michael de Marco, Weinheim (DE); Patrick Deck, Mannheim (DE); Oliver Huttenloch, Ispringen (DE); Michael Mauss, Neustadt (DE)

Correspondence Address: MARSHALL, GERSTEIN & BORUN LLP, 233 S. WACKER DRIVE, SUITE 6300, SEARS TOWER, CHICAGO, IL, 60606, US

(21) Appl. No.: 11/993,069

(22) PCT Filed: Jul. 18, 2006
(86) PCT No.: PCT/EP2006/064354

§ 371 Date: Dec. 19, 2007

Related U.S. Application Data
(60) Provisional application No. 60/702,931, filed on Jul. 27, 2005.

(51) Int. Cl. A61F 13/53; C08K 5/5399

(52) U.S. Cl. 604/372; 524/146

(57) ABSTRACT

The present invention leads to odor-preventing water-absorbing compositions comprising at least one water-absorbing polymer and at least one substituted thiophosphoramide, to processes for their production and also to hygiene articles and their production.

The present invention leads to odor-preventing water-absorbing compositions comprising at least one water-absorbing polymer and at least one substituted thiophosphoramide, to processes for their production and also to hygiene articles and their production.

Further embodiments of the present invention are discernible from the claims, the description and the examples. It will be understood that the hereinbefore mentioned and the hereinbelow still to be elucidated features of the present invention's subject matter are utilizable not only in the particular combination indicated but also in other combinations without leaving the realm of the invention.

Water-absorbing polymers are in particular polymers of (co)polymerized hydrophilic monomers, graft (co)polymers of one or more hydrophilic monomers on a suitable grafting base, crosslinked ethers of cellulose or starch, crosslinked carboxymethylcellulose, partially crosslinked polyalkylene oxide or natural products swellable in aqueous fluids, examples being guar derivatives, water-absorbing polymers based on partially neutralized acrylic acid being preferred. Such polymers are used as products capable of absorbing aqueous solutions to manufacture diapers, tampons, sanitary napkins, incontinence products and other hygiene articles, but also as water-retaining agents in market gardening.

Unpleasant odors can arise in hygiene articles during use through decomposition of urea for example. WO-A-98126808, WO-A-03/053486, EP-A-0 739 635, EP-A-1 034 800 and EP-A-1 214 878 propose various solutions to the problem.

WO-A-98/26808 describes absorbent compositions comprising a fluid-absorbing material, an odor-absorbing material and also one or more substances from the group consisting of biocides, urease inhibitors and pH regulators.

WO-A-03/053486 discloses the use of yucca extract as a urease inhibitor.

EP-A-0 739 635 describes absorbent compositions comprising boric acid salts.

EP-A-1 034 800 describes the use of combinations of an odor-absorbing agent and an oxidizing agent to control unpleasant odors.

EP-A-1 214 878 teaches the use of metal chelates as urease inhibitors.

The present invention has for its object to provide improved water-absorbing compositions which, having been insulted with urine or other body fluids, reliably prevent unpleasant odors. Since the use of biocides is problematic directly in contact with the skin, the compositions must not comprise any significant biocidal effect either.

The present invention further has for its object to provide odor-preventing water-absorbing compositions which are stable in storage, i.e., which neither discolor nor lose their odor-preventing effect in the course of prolonged storage.

We have found that this object is achieved by compositions comprising at least one water-absorbing polymer and at least one substituted thiophosphoramide of the formula (I)

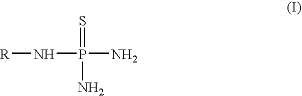

where R is a C₁- to C₃₀-alkyl radical, preferably a C₂- to C₁₀-alkyl radical and more preferably a C₃- to C₅-alkyl radical. The alkyl radicals may be branched or unbranched.

The examples of C₁- to C₁₀-alkyl radicals are methyl, ethyl, n-propyl, isopropyl, n-butyl, isobutyl, sec-butyl, tert-butyl, n-pentyl, isopentyl, n-hexyl, isohexyl, n-heptyl, isoheptyl, n-octyl, isooctyl, n-nonyl, isononyl, n-decyl and isodecyl. The most preferred alkyl radicals are n-propyl and n-butyl.

The composition of the present invention comprises typically from 0.0001% to 5% by weight, preferably from 0.003% to 1% by weight and more preferably from 0.005% to 0.1% by weight of the at least one substituted thiophosphoramide.

The at least one water-absorbing polymer is preferably a polymer based on a partially neutralized crosslinked acrylic acid.

The composition of the present invention comprises typically at least 90% by weight, preferably at least 95% by weight, and more preferably at least 99% by weight of the at least one water-absorbing polymer.

The substituted thiophosphoramides of the formula (I) are obtainable for example by reacting thiophosphoryl trichloride with alkylamine and ammonia.

The preparation of substituted thiophosphoramides is described in U.S. Pat. No. 5,770,771 for example. Higher purities are achieved when the products are recrystallized in a suitable solvent, an example being toluene.

The water-absorbing polymers are obtained for example by polymerization of a monomer solution comprising
a) at least one ethylenically unsaturated acid-functional monomer,
b) at least one crosslinker,
c) if appropriate one or more ethylenically and/or allylically unsaturated monomers copolymerizable with the monomer a), and
d) if appropriate one or more water-soluble polymers onto which the monomers a), b) and if appropriate c) can be at least partly grafted.

Suitable monomers a) are for example ethylenically unsaturated carboxylic acids, such as acrylic acid, methacrylic acid, maleic acid, fumaric acid and itaconic acid, or derivatives thereof, such as acrylamide, methacrylamide, acrylic esters and methacrylic esters. Acrylic acid and methacrylic acid are particularly preferred. Acrylic acid is most preferable.

The monomers a) and especially acrylic acid comprise preferably up to 0.025% by weight of a hydroquinone half ether. Preferred hydroquinone half ethers are hydroquinone monomethyl ether (MEHQ) and/or tocopherols.

Tocopherol refers to compounds of the following formula:

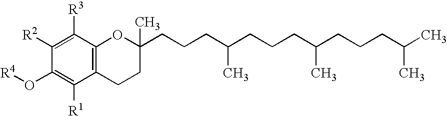

where R¹ is hydrogen or methyl, R² is hydrogen or methyl, R³ is hydrogen or methyl and R⁴ is hydrogen or an acyl radical of 1 to 20 carbon atoms.

Preferred R⁴ radicals are acetyl, ascorbyl, succinyl, nicotinyl and other physiologically tolerable carboxylic acids. The carboxylic acids can be mono-, di- or tricarboxylic acids.

Preference is given to alpha-tocopherol where R¹=R²=R³=methyl, especially racemic alpha-tocopherol. R¹ is more preferably hydrogen or acetyl. RRR-alpha-tocopherol is preferred in particular.

The monomer solution comprises preferably not more than 130 weight ppm, more preferably not more than 70 weight ppm, preferably not less than 10 weight ppm, more preferably not less than 30 weight ppm and especially about 50 weight ppm of hydroquinone half ether, all based on acrylic acid, with acrylic acid salts being counted as acrylic acid. For example, the monomer solution can be produced using an acrylic acid having an appropriate hydroquinone half ether content.

The crosslinkers b) are compounds having at least two polymerizable groups which can be free-radically interpolymerized into the polymer network. Suitable crosslinkers b) are for example ethylene glycol dimethacrylate, diethylene glycol diacrylate, allyl methacrylate, trimethylolpropane triacrylate, triallylamine, tetraallyloxyethane, as described in EP-A-0 530 438, di- and triacrylates, as described in EP-A-0 547 847, EP-A-0 559 476, EP-A-0 632 068, WO-A-93/21237, WO-A-03/104299, WO-A-03/104300, WO-A-03/104301 and DE-A-103 31 450, mixed acrylates which, as well as acrylate groups, comprise further ethylenically unsaturated groups, as described in DE-A-103 31 456 and WO-A-04/013064, or crosslinker mixtures as described for example in DE-A-195 43 368, DE-A-196 46 484, WO-A-90/15830 and WO-A-02/32962.

Useful crosslinkers b) include in particular N,N′-methylenebisacrylamide and N,N′-methylenebismethacrylamide, esters of unsaturated mono- or polycarboxylic acids of polyols, such as diacrylate or triacrylate, for example butanediol diacrylate, butanediol dimethacrylate, ethylene glycol diacrylate, ethylene glycol dimethacrylate and also trimethylolpropane triacrylate and allyl compounds, such as allyl (meth)acrylate, triallyl cyanurate, diallyl maleate, polyallyl esters, tetraallyloxyethane, triallylamine, tetraallylethylenediamine, allyl esters of phosphoric acid and also vinylphosphonic acid derivatives as described for example in EP-A-0 343 427. Useful crosslinkers b) further include pentaerythritol diallyl ether, pentaerythritol triallyl ether, pentaerythritol tetraallyl ether, polyethylene glycol diallyl ether, ethylene glycol diallyl ether, glycerol diallyl ether, glycerol triallyl ether, polyallyl ethers based on sorbitol, and also ethoxylated variants thereof. The process of the invention utilizes di(meth)acrylates of polyethylene glycols, the polyethylene glycol used having a molecular weight between 300 and 1000.

However, particularly advantageous crosslinkers b) are di- and triacrylates of 3- to 15-tuply ethoxylated glycerol, of 3- to 15-tuply ethoxylated trimethylolpropane, of 3- to 15-tuply ethoxylated trimethylolethane, especially di- and triacrylates of 2- to 6-tuply ethoxylated glycerol or of 2- to 6-tuply ethoxylated trimethylolpropane, of 3-tuply propoxylated glycerol, of 3-tuply propoxylated trimethylolpropane, and also of 3-tuply mixedly ethoxylated or propoxylated glycerol, of 3-tuply mixedly ethoxylated or propoxylated trimethylolpropane, of 15-tuply ethoxylated glycerol, of 15-tuply ethoxylated trimethylolpropane, of 40-tuply ethoxylated glycerol, of 40-tuply ethoxylated trimethylolethane and also of 40-tuply ethoxylated trimethylolpropane.

Very particularly preferred for use as crosslinkers b) are diacrylated, dimethacrylated, triacrylated or trimethacrylated multiply ethoxylated and/or propoxylated glycerols as described for example in WO-A-03/104301. Di- and/or triacrylates of 3- to 10-tuply ethoxylated glycerol are particularly advantageous. Very particular preference is given to di- or triacrylates of 1- to 5-tuply ethoxylated and/or propoxylated glycerol. The triacrylates of 3- to 5-tuply ethoxylated and/or propoxylated glycerol are most preferred. These are notable for particularly low residual levels (typically below 10 weight ppm) in the water-absorbing polymer and the aqueous extracts of water-absorbing polymers produced therewith have an almost unchanged surface tension (typically not less than 0.068 N/m) compared with water at the same temperature.

The amount of crosslinker b) is preferably from 0.01 to 1% by weight, more preferably from 0.05 to 0.5% by weight and most preferably from 0.1 to 0.3% by weight, all based on a monomer a).

Examples of ethylenically unsaturated monomers c) which are copolymerizable with the monomers a) are acrylamide, methacrylamide, crotonamide, dimethylaminoethyl methacrylate, dimethylaminoethyl acrylate, dimethylaminopropyl acrylate, diethylaminopropyl acrylate, dimethylaminobutyl acrylate, dimethylaminoethyl methacrylate, diethylaminoethyl methacrylate, dimethylaminoneopentyl acrylate and dimethylaminoneopentyl methacrylate.

Useful water-soluble polymers d) include polyvinyl alcohol, polyvinylpyrrolidone, starch, starch derivatives, polyglycols or polyacrylic acids, preferably polyvinyl alcohol and starch.

Polymerization inhibitors, which are preferred, require dissolved oxygen for optimum performance. Therefore, polymerization inhibitors may be freed of dissolved oxygen prior to polymerization by inertization, i.e, flowing an inert gas, preferably nitrogen, through them. The oxygen content of the monomer solution is preferably lowered to less than 1 weight ppm and more preferably to less than 0.5 weight ppm prior to polymerization.

The preparation of a suitable base polymer and also further useful hydrophilic ethylenically unsaturated monomers d) are described in DE-A-199 41 423, EP-A-0 686 650, WO-A-01/45758 and WO-A-03/104300.

Water-absorbing polymers are typically obtained by addition polymerization of an aqueous monomer solution with or without subsequent comminution of the hydrogel. Suitable methods of making are described in the literature. Water-absorbing polymers are obtainable for example by
gel polymerization in the batch process or tubular reactor and subsequent comminution in meat grinder, extruder or kneader (EP-A-0 445 619, DE-A-19 846 413)
addition polymerization in kneader with continuous comminution by contrarotatory stirring shafts for example (WO-A-01/38402)
addition polymerization on belt and subsequent comminution in meat grinder, extruder or kneader (DE-A-38 25 366, U.S. Pat. No. 6,241,928)
emulsion polymerization, which produces bead polymers having a relatively narrow gel size distribution (EP-A-0 457 660)
in situ addition polymerization of a woven fabric layer which, usually in a continuous operation, has previously been sprayed with aqueous monomer solution and subsequently been subjected to a photopolymerization (WO-A-02194328, WO-A-02/94329).

The reaction is preferably carried out in a kneader as described for example in WO-A-01/38402, or on a belt reactor as described for example in EP-A-0 955 086.

The acid groups of the hydrogels obtained have typically been partially neutralized, preferably to an extent of in the range from 25 to 85 mol %, more preferably to an extent of in the range from 27 to 80 mol %, even more preferably to an extent of in the range from 27 to 30 mol % or 40 to 75 mol %, and most preferably to an extent of in the range from 50 to 65 mol %, for which the customary neutralizing agents can be used, preferably alkali metal hydroxides, alkali metal oxides, alkali metal carbonates or alkali metal bicarbonates and also mixtures thereof. Instead of alkali metal salts it is also possible to use ammonium salts. Sodium and potassium are particularly preferred as alkali metals, but most preference is given to sodium hydroxide, sodium carbonate or sodium bicarbonate and also mixtures thereof. Neutralization is customarily achieved by admixing the neutralizing agent as an aqueous solution or else preferably as a solid material. For example, sodium hydroxide having a water content of distinctly below 50% by weight can be present as a waxy mass having a melting point of above 23° C. In this case, metering as piecegoods or melt at elevated temperature is possible.

Neutralization can be carried out after polymerization, at the hydrogel stage. But it is also possible to neutralize up to 40 mol %, preferably from 10 to 30 mol % and more preferably from 15 to 25 mol % of the acid groups before polymerization by adding a portion of the neutralizing agent to the monomer solution and setting the desired final degree of neutralization only after polymerization, at the hydrogel stage. The monomer solution can be neutralized by admixing the neutralizing agent. The hydrogel may be mechanically comminuted, for example by means of a meat grinder, in which case the neutralizing agent can be sprayed, sprinkled or poured on and then carefully mixed in. To this end, the gel mass obtained can be repeatedly meat-grindered for homogenization. Neutralization of the monomer solution to the final degree of neutralization is preferred.

The neutralized hydrogel is then dried with a belt or drum dryer until the residual moisture content is preferably below 15% by weight and especially below 10% by weight, the water content being determined by EDANA (European Disposables and Nonwovens Association) recommended test method No. 430.2-02 “Moisture content”. Selectively, drying can also be carried out using a fluidized bed dryer or a heated plowshare mixer. To obtain particularly white products, it is advantageous to dry this gel by ensuring rapid removal of the evaporating water. To this end, the dryer temperature must be optimized, the air feed and removal has to be policed, and at all times sufficient venting must be ensured. Drying is naturally all the more simple—and the product all the more white—when the solids content of the gel is as high as possible. The solids content of the gel prior to drying is therefore preferably between 30% and 80% by weight. It is particularly advantageous to vent the dryer with nitrogen or some other nonoxidizing inert gas. Selectively, however, simply just the partial pressure of the oxygen can be lowered during drying to prevent oxidative yellowing processes. But in general adequate venting and removal of the water vapor will likewise still lead to an acceptable product. A very short drying time is generally advantageous with regard to color and product quality.

The dried hydrogel is preferably ground and sieved, useful grinding apparatus typically including roll mills, pin mills or swing mills. The particle size of the sieved, dry hydrogel is preferably below 1000 μm, more preferably below 900 μm and most preferably below 850 μm and preferably above 80 μm, more preferably above 90 μm and most preferably above 100 μm.

Very particular preference is given to a particle size (sieve cut) in the range from 106 to 850 μm. The particle size is determined according to EDANA (European Disposables and Nonwovens Association) recommended test method No. 420.2-02 “Particle size distribution”.

The base polymers are then preferably surface postcrosslinked. Useful postcrosslinkers are compounds comprising two or more groups capable of forming covalent bonds with the carboxylate groups of the hydrogel. Suitable compounds are for example alkoxysilyl compounds, polyaziridines, polyamines, polyamidoamines, di- or polyepoxides, as described in EP-A-0 083 022, EP-A-543 303 and EP-A-937 736, di- or polyfunctional alcohols, as described in DE-C-33 14 019, DE-C-35 23 617 and EP-A-450 922, or 1′-hydroxyalkylamides, as described in DE-A-102 04 938 and U.S. Pat. No. 6,239,230.

Useful surface postcrosslinkers are further said to include by DE-A-40 20 780 cyclic carbonates, by DE-A-1 98 07 502 2-oxazolidone and its derivatives, such as 2-hydroxyethyl-2-oxazolidone, by DE-A-198 07 992 bis- and poly-2-oxazolidinones, by DE-A-1 98 54 573 2-oxotetrahydro-1,3-oxazine and its derivatives, by DE-A-1 98 54 574 N-acyl-2-oxazolidones, by DE-A-102 04 937 cyclic ureas, by DE-A-103 34 584 bicyclic amide acetals, by EP-A-1 199 327 oxetanes and cyclic ureas and by WO-A-03/031482 morpholine-2,3-dione and its derivatives.

It is advantageous to use polyvalent cations for surface postcrosslinking as well as surface postcrosslinkers. Useful polyvalent cations include for example divalent cations, such as the cations of zinc, magnesium, calcium and strontium, trivalent cations, such as the cations of aluminum, iron, chromium, rare earths and manganese, tetravalent cations, such as the cations of titanium and zirconium. Possible counterions are chloride, bromide, sulfate, hydrogen sulfate, carbonate, hydrogencarbonate, nitrate, phosphate, hydrogen phosphate, dihydrogen phosphate and carboxylate, such as acetate and lactate. Aluminum sulfate is preferred.

Postcrosslinking is typically carried out by spraying a solution of the surface postcrosslinker onto the hydrogel or onto the dry base-polymeric powder. The surface postcrosslinker and the polyvalent cation can be sprayed in a common solution or as separate solutions. After spraying, the polymeric powder is thermally dried, and the crosslinking reaction may take place not only before but also during drying.

The spraying with a solution of the crosslinker is preferably carried out in mixers having moving mixing implements, such as screw mixers, paddle mixers, disk mixers, plowshare mixers and shovel mixers. Particular preference is given to vertical mixers and very particular preference to plowshare mixers and shovel mixers. Useful mixers include for example Lödige® mixers, Bepex® mixers, Nauta® mixers, Processall® mixers and Schugi® mixers. Very particular preference is given to employing high-speed mixers, for example of the Schuggi-Flexomix® or Turbolizer® type.

Contact dryers are preferable, shovel dryers more preferable and disk dryers most preferable as apparatus in which thermal drying is carried out. Useful dryers include for example Bepex® dryers and Nara® dryers. Fluidized bed dryers can be used as well.

Drying may take place in the mixer itself, by heating the jacket or introducing a stream of warm air. It is similarly possible to use a downstream dryer, for example a tray dryer, a rotary tube oven or a heatable screw. But it is also possible for example to utilize an azeotropic distillation as a drying process.

Preferred drying temperatures are in the range from 50 to 250° C., preferably in the range from 50 to 200° C. and more preferably in the range from 50 to 150° C. The preferred residence time at this temperature in the reaction mixer or dryer is below 30 minutes and more preferably below 10 minutes.

The present invention further provides processes for producing the compositions of the present invention, said processes comprising
i) mixing at least one substituted thiophosphoramide with at least one water-absorbing polymer, and/or
ii) grinding at least one substituted thiophosphoramide together with at least one water-absorbing polymer, and/or
iii) spraying at least one substituted thiophosphoramide onto at least one water-absorbing polymer, and/or
iv) preparing the at least one water-absorbing polymer by solution polymerization of a monomer solution and dissolving or suspending at least one substituted thiophosphoramide in the monomer solution,
   and optionally mixing the composition obtained according to i), ii), iii) and/or iv) together with at least one water-absorbing polymer.

Mixing may be carried out in any manner and may be effected as early as the production of the water-absorbing polymer, for example in the course of cooling after postcrosslinking or the subsequent sieving, or in a special mixer. Suitable mixers were described above in relation to the postcrosslinking of the water-absorbing polymer.

The manner of grinding is likewise not subject to any restriction. Suitable apparatuses were described above in relation to the comminution of the water-absorbing polymer.

The manner of spraying is not subject to any restriction. The substituted thiophosphoramide may be sprayed as a solution or as a melt, for example during the postcrosslinking of the water-absorbing polymer in the mixers mentioned there.

The at least one substituted thiophosphoramide is advantageously sprayed as a solution in a suitable solvent. Suitable solvents are water, water-acetone mixtures, water-propylene glycol mixtures and also the solvents and solvent mixtures identified in relation to the postcrosslinking operation. The concentration of the substituted thiophosphoramide in the solution is typically in the range from 0.5% to 30% by weight, preferably in the range from 1% to 20% by weight and more preferably in the range from 2% to 10% by weight.

A further embodiment comprises producing a composition according to the present invention that comprises a higher fraction of the at least one substituted thiophosphoramide, typically in the range from 1% to 50% by weight, preferably in the range from 5% to 40% by weight and more preferably in the range from 10% to 30% by weight. The highly concentrated composition thus obtained may then be diluted with further water-absorbing polymer to the desired final strength.

The present invention further provides hygiene articles comprising at least one composition according to the present invention, in particular diapers or pads for heavy and/or light incontinence and also sanitary napkins, and processes for producing hygiene articles wherein at least one composition according to the present invention is used.

The water-absorbing compositions of the present invention are able to reliably prevent unpleasant odors which can arise in hygiene articles. The compositions of the present invention are stable on storage, so that the odor-controlling effect is still present after prolonged storage, for example 6 months. Furthermore, the compositions of the present invention are free of visible discolorations after prolonged storage.

### EXAMPLES

### Example 1

### Production of Water-Absorbing Polymer

4809 g of a 37.3% by weight aqueous sodium acrylate solution were mixed with 534 g of acrylic acid and 573 g of water and inertized with nitrogen. This mixture was filled into a nitrogen-inertized Werner & Pfleiderer LUK 8,0 K2 kneader (2 sigma shafts) and admixed in succession with 4.8 g of polyethylene glycol diacrylate 400 (diacrylate of a polyethylene glycol having an average molecular weight of 400 g/mol), 4.8 g of 15-tuply ethoxylated trimethylolpropane triacrylate, 4.4 g of a 1.0% by weight aqueous ascorbic acid solution, 18.1 g of a 15% by weight aqueous sodium persulfate solution and 3.9 g of a 3% by weight aqueous hydrogen peroxide solution. The kneader was stirred at maximum speed (98 rpm for the faster shaft, about 49 rpm for the slower shaft, ratio about 2:1). Immediately following the addition of hydrogen peroxide, the kneader jacket was heated with hot heat transfer medium at 80° C. On reaching the maximum temperature, the jacket heating was switched off and the kneader contents were allowed to react for a further 15 minutes in a supplementary reaction. The gel was cooled down to 65° C. and discharged. The gel was dried at 175° C. for 75 minutes using a loading of 700 g per tray in a circulating air drying cabinet. Following threefold grinding in a roll mill (Gebr. Baumeister LRC 125/70, gap widths 1000 μm, 600 μm, 400 μm), the polymer was sieved to obtain a size cut between 850 and 100 μm.

1200 g of this polymer were transferred into a Gebr. Lödige laboratory mixer (M5R model). At room temperature, a mixture of 12 g of 1,2-propanediol, 1.3 g of diethylene glycol diglycidyl ether and 28 g of water was sprayed in via a first nozzle and 12 g of an aluminum sulfate solution (26.8% by weight of Al₂(SO₄)₃ in water) via a second nozzle. The mixer was then rapidly heated to 168° C. and maintained at 168° C. for 40 minutes. After cooling, the polymer was sieved to obtain a size cut between 850 and 100 μm.

### Example 2

100 g lots of water-absorbing polymer from Example 1 were mixed in a tumble mixer with different amounts of N-cyclohexylthiophosphoramide, N-(n-butyl)thiophosphoramide or N-(n-propyl)thiophosphoramide, for 20 minutes each.

To determine the odor-preventing effect, 2 g of each of the compositions produced above were placed in a 100 ml Erlenmeyer flask and admixed with a freshly prepared solution of 30 mg of urease (from jack beans; lyophilized 5 U/mg for urea assay in serum; Merck KGaA, Germany) and 50 ml of 0.9% sodium chloride solution, the sodium chloride solution containing 8.56 g/l of urea, and sealed with a stopper having an internal diffusion tublet (Drager® Röhrchen; ammonia 20/a-D, 20 to 1500 ppm*h). The measured value was read off every 30 minutes. The measurement was discontinued after 6 hours. The test was carried out at 23° C. The following table shows the measured results:

TABLE 1

| | Concentration of substituted | | | | | | |
|---|---|---|---|---|---|---|---|
| | thiophosphoramide in composition | | | | | | |
| | [% by weight] | | | | | | |
| | 1.50 | 0.50 | 0.35 | 0.25 | 0.15 | 0.05 | 0.01 |
| R = cyclohexyl | + | + | + | − | − | − | − |
| R = n-butyl | + | + | + | + | + | + | + |
| R = n-propyl | + | + | + | + | + | + | + |

+ less than 50 ppm of ammonia after 6 hours
− more than 50 ppm of ammonia after 6 hours

The compositions had no significant bactericidal effect on Escherichia coli, Staphylococcus aureus and Proteus mirabilis.

1. A composition comprising at least one water-absorbing polymer and at least one substituted thiophosphoramide of a formula (I) 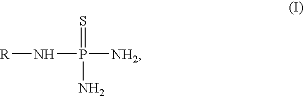
wherein R is a C₁- to C₃₀-alkyl radical.

2. The composition according to claim 1 comprising 0.0001% to 5% by weight of the substituted thiophosphoramide.

3. The composition according to claim 1 comprising at least 90% by weight of the water-absorbing polymer.

4. The composition according to claim 1 wherein the alkyl radical R is n-propyl or n-butyl.

5. The composition according to claim 1 wherein the water-absorbing polymer is based on a partially neutralized crosslinked acrylic acid.

6. The composition according to claim 1 wherein the water-absorbing polymer is surface postcrosslinked.

7. A process for producing a composition as defined in claim 1, which comprises performing at least one of the following steps:
i) mixing the at least one substituted thiophosphoramide with the at least one water-absorbing polymer, and/or
ii) grinding the at least one substituted thiophosphoramide together with the at least one water-absorbing polymer, and/or
iii) spraying the at least one substituted thiophosphoramide onto the at least one water-absorbing polymer, and/or
iv) preparing the at least one water-absorbing polymer by solution polymerization of a monomer solution and dissolving or suspending the at least one substituted thiophosphoramide in the monomer solution,
and optionally mixing a composition obtained according to i), ii), iii) and/or iv) together with the at least one water-absorbing polymer.

8. A hygiene article comprising at least one composition according to claim 1.

9. The hygiene article according to claim 8 being a diaper or a pad for heavy and/or light incontinence.

10. (canceled)
